(19) United States
(12) Patent Application Publication
(10) Pub. No.: US 2016/0199235 A1
(43) Pub. Date: Jul. 14, 2016

(54) WATER-ABSORBENT MATERIAL WITH ADJUSTABLE DESALINATION FUNCTION

(71) Applicant: BASF SE, Ludwigshafen (DE)

(72) Inventors: Thomas Daniel, Waldsee (DE); Xiaomin Zhang, Charlotte, NC (US); Michael Mitchell, Waxhaw, NC (US)

(21) Appl. No.: 14/772,812

(22) PCT Filed: Mar. 3, 2014
(86) PCT No.: PCT/EP2014/054035

§ 371 Date: Sep. 4, 2015

Related U.S. Application Data
(60) Provisional application No. 61/778,452, filed on Mar. 13, 2013.

(51) Int. Cl. A61F 13/53; B29C 47/00; A61F 13/15

(52) U.S. Cl. CPC A61F 13/53; A61F 13/15617; B29C 47/0004; A61F 2013/530226; B29K 2025/08

(57) ABSTRACT

The present invention relates to water absorbing material comprising acidic water-absorbent electrospun fibres and basic water-absorbent electrospun fibres, wherein the fibres are at least partially in contact to each other and a process for producing water absorbing material.

The present invention relates to water absorbing material comprising acidic water-absorbent electrospun fibres and basic water-absorbent electrospun fibres, wherein the fibres are at least partially in contact to each other and a process for producing water absorbing material.

Water-absorbing polymers (“super absorbent polymer”, SAP) are widely used in sanitary goods and hygiene articles such as disposable diapers, adult incontinence pads and catamenial products as sanitary napkins. Water absorbing resins are available in a variety of chemical forms. The production of SAP particles is described in the monograph “Modern Superabsorbent Polymer Technology”, F. L. Buchholz and A. T. Graham, Wiley-VCH, 1998, pages 71 to 103.

Generally the ability to absorb aqueous fluids under confining pressure is an important requirement for SAP to be used in hygiene articles. As the swelling and absorbent properties of the SAPs are attributed to electrostatic repulsion between the charges along the polymer chains, and osmotic pressure of the counter ions, these absorption properties are drastically reduced in solutions containing electrolytes, such as saline, urine, blood. The polymer functions much less effectively in the presence of such physiologic fluids (“salt poisoning”). The commonly used SAP for absorbing electrolyte-containing liquids, such as urine, is neutralized polyacrylic acid, i.e. containing at least 50% and up to 100% neutralized carboxyl-groups. Neutralized polyacrylic acid, however, is susceptible in salt poisoning.

Much effort is taken to very thin absorbent articles such as diapers, because they fit better and less noticeable and as article packaging is more compact they are easier to carry and to store and therefore the distribution costs are reduced Therefore many attempts in reducing the amount of SAP particles in such products and reduction of fluff are made, but as gel blocking has necessitated the use of a fibrous matrix to disperse the SAP particles and separate the SAP particles from one another the reduction of fluff causes a lot of problems.

Furthermore the SAP particles e.g. tend to move from where they are placed. Therefore additional handling and manufacturing steps are necessary such as the SAP particles must be glued, fused or laminated to a support structure. To overcome this SAP particles in fiber form are developed. For example U.S. Pat. Nos. 5,147,956 and 4,962,172 disclose absorbent products and their method of manufacture.

Also special SAP's in fiber form have been developed with an improved absorption for electrolyte-containing liquids. Such multicomponent SAPs comprising at least one acidic water-absorbing resin, such as polyacrylic acid, covalently bound to at least one basic water-absorbing resin, such as a poly(vinylamine) or a polyethyleneimine, utilizing an interfacial crosslinking agent. The acidic resin removes the sodium ion; and the basic resin removes the chloride ions. This ion exchange reaction, therefore, produces water as sodium chloride is adsorbed onto the resins (“desalination function”).

U.S. Pat. No. 6,342,298 and U.S. Pat. No. 6,376,072 disclose a method for producing multicomponent SAP fibers. The fibers are prepared by gel extrusion or dry or wet spinning. The multicomponent fibers comprise at least one acidic water-absorbing resin and at least one basic water-absorbing resin. Each fiber contains at least one microdomain of the acidic resin in contact with, or in close proximity to, at least one microdomain of the basic resin.

Various further references (e.g. WO 98/24832, US 2003/0138631, WO 99/025393) disclose combinations that attempt to overcome the salt poisoning effect.

However, these mixed superabsorbents particles and fibres are very cost intensive especially because of the manufacturing process, which comprises many different steps such as mixing dry particles of a basic resin with a rubbery gel of an acidic resin. Then the mixture has to be extruded and dried.

As the number and the alignment of basic and acidic resins in the fiber can only indirectly be influenced by the amounts of each resin used the desalination function and therefore the fluid absorption and retention properties of the fibres vary. It is not possible to adjust these properties.

Therefore it is one object of the present invention to provide a costly water-absorbent material with a high capacity for “charged” solutions.

It is also an object of the present invention to provide water-absorbent material with an adjustable desalination function. The fluid absorption and retention properties of the material could easily be adapted for different uses.

It is also is an object of the present invention to reduce the amount of superabsorbent material in different applications by providing tailored solutions.

Furthermore it is an object of the present invention to provide a cost efficient production process for such superabsorbent material.

It is also an object of the present invention to provide hygiene products, fluid-absorbent articles with improved fluid absorption and retention properties.

The object is achieved by water absorbing material comprising acidic electrospun fibres and basic electrospun fibres, wherein the fibres are at least partially in contact to each other.

The acidic electrospun fibres of the inventive water absorbing material comprise
at least one water-soluble acidic functional polymer and
at least one crosslinker,
and
   the basic electrospun fibres comprise
at least one water-soluble basic functional polymer and
at least one crosslinker.

It is preferable that the water-soluble polymers are non-neutralized.

The inventive water absorbing material provides improved fluid absorption especially in the amount of electrolyte containing fluids absorbed and retained and in the rate of absorption even compared to multicomponent SAP fibers known in the art.

In the inventive water absorbing material the electrospun fibres are preferably arranged in layers.

Layers are concerned in the present invention. The meaning which is assigned to layers in connection with this invention is the usual one of a shaped article which has distinctly less extent in one of three dimensions of a cartesian system of coordinates than in the other two dimensions. “Thickness” in connection with this invention is to be understood as meaning that of these dimensions in which the layer has the least extent. Put simply, layers are longer and wider than they are thick.

Whereas the layers of the water-absorbing electrospun fibres can be of any size, dependent on the expected use. Especially for use in diapers the layers could be formed to exactly fit in the shape of a diaper.

The thickness of each layer is not greater than 5 μm, preferably not more than 3 μm, more preferably not greater than 1 μm, most preferably not greater than 0.3 μm, but it is also possible that the thickness of the layer is not greater than 0.1 μm.

Minimum thickness is solely determined by the needed mechanical stability of the layer and is chosen accordingly. What is generally sufficient is a thickness of at least 3 μm, preferably at least 1 μm.

The preferred thickness of the layer preferably is of at least 1 μm.

The layers could contain only acidic or only basic electrospun fibres. But it is also possible that both fibres are mixed.

Wherein it is preferred that the acidic electrospun fibres are arranged in at least one acidic layer and the basic electrospun fibres are arranged in at least one basic layer and the layers are at least partially overlapping.

The different layers of the water-absorbing material are preferably attached to each other directly without any further substance such as an adhesive.

Preferably within the water absorbing material the basic-layers (B) and acidic-layers (A) are arranged according to any suitable pattern.

According to one preferred embodiment the water absorbing material comprises layers, which are all of comparable size.

It may be preferable that the water absorbing material comprises layers, which are of comparable size, except for the thickness.

Whereas the thickness of the water absorbing material also can be of any size, dependent on the expected use. For use in diapers the thickness preferably is in the range of 2 μm to 15 μm, more preferably 3 μm to 10 μm.

For use as filters suitable for desalination of fluids the thickness of the material may be preferred 0.05 μm to 2 μm, more preferably from 0.1 μm to 1 μm.

It may be preferable that the layers are arranged in a sandwich like structure, wherein each layer overlaps the preceding and/or succeeding layer by more than 80%, preferably 85, more preferably more than 90% of its area.

Whereas it is preferred that in a sandwich like structure the layers are arranged alternating, e.g. ABA . . . , or AABAA . . . , or BBABBA . . . , AABBAABB . . . , AAABBB . . . or any other suitable patterns.

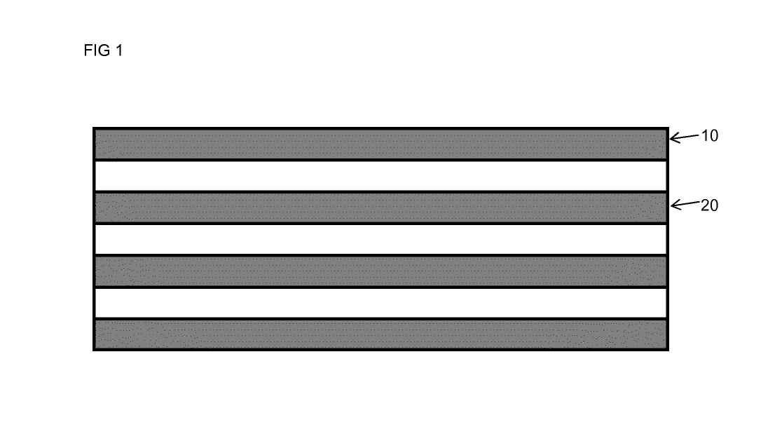

FIG. 1 are a cross section of an absorbing material containing 100% by weight layers of the present invention. The layers are arranged alternating acidic layer (10) and basic layer (20).

The weight ratio of acidic water-absorbing electrospun fibres to basic water-absorbing electrospun fibres in the water-absorbing material is of about 95:5 to about 5:95, preferably of 30:70 to 70:30, more preferably 40:60 to 60:40. To achieve the full advantage of the present invention, the weight ratio of the acidic fibres to basic fibres in the absorbing material is about 50:50. Furthermore the inventive water absorbing material provides a very high desalination index.

The desalination index (DI), for each absorbing material is given by

$$DI = \frac{Absorbent capacity in saline}{Absorbent Capacity in deionized water}$$

The desalination index for the inventive water absorbent material is at least 0.8, preferable at least 0.9, more preferable at least 1, and most preferable at least 1.05.

Preferable the absorbent capacity of the water absorbent material for electrolyte containing solutions is at least the same more preferable higher than for deionized water.

The inventive water absorbing material preferably has an absorbent capacity in saline of at least 10 g/g, more preferable at least 12 g/g, most preferable at least 15 g/g.

The desalination index depends on the weight ratio of acidic water-absorbing electrospun fibres to basic water-absorbing electrospun fibres and the number of layers the absorbing material comprises, and on the arrangement of the layers, e.g. in sandwich structure if the layers are arranged alternating or according to a different pattern.

For achieving a high desalination index it is preferable that acidic and basic layers are arranged in a sandwich like structure, comprising layers arranged alternating (ABABAB, or BABABA). Furthermore it is preferred for a Desalination index of at least 1 that the water absorbing material comprises more than 3 layers.

The Desalination Index (DI) of the water absorbing material according to the invention is of at least 0.85, preferably at least 0.90 more preferably at least 1.0, most preferably at least 1.10.

A comparatively simple process for producing the water-absorbent materials of the present invention comprises the step of
preparing at least one layer of electrospun fibres by
a) electrospinning a solution of at least one acidic water-soluble polymer or of at least one basic water-soluble polymer and at least one crosslinker,
b) collecting the fibres on a substrate,
c) optionally repeating a) to b) at least once
d) crosslinking the fibres
e) optionally repeating a) to d).

One embodiment of the water-absorbent materials of the present invention could be produced by a process comprising
preparing at least one layer of electrospun fibres by
a) electrospinning a solution of at least one acidic water-soluble polymer or of at least one basic water-soluble polymer and at least one crosslinker,
b) collecting the fibres on a substrate,
c) repeating a) to b) at least once
d) crosslinking the fibres
e) optionally repeating a) to d).

A further embodiment of the water-absorbent materials of the present invention could be produced by a process comprising
preparing at least one layer of electrospun fibres by
a) electrospinning a solution of at least one acidic water-soluble polymer or of at least one basic water-soluble polymer and at least one crosslinker,
b) collecting the fibres on a substrate,
c) optionally repeating a) to b) at least once
d) crosslinking the fibres
e) repeating a) to d).

Any method that provides an electrospun water-absorbent fiber is suitable.

The preparation of polymer fibers, especially nano- and mesofibers by the electrospinning process is well described in various documents.

The process is e.g. described by D. H. Renecker, H. D. in Nanotech. 7 (1996), page 216 ff. A polymer melt or a polymer solution is typically exposed to a high electrical field at an edge which serves as an electrode. This can be achieved e. g. by extrusion of the polymer melt or polymer solution in an electrical field under low pressure by a cannula connected to one pole of a voltage source. Owing to the resulting electrostatic charge of the polymer melt or polymer solution, there is a material flow directed toward the counterelectrode, which solidifies on the way to the counterelectrode. Depending on the geometry of the electrode nonwovens or assemblies of ordered fibers are obtained by this process.

US 2010/0013126 discloses the electrospinning of at least one essentially water-insoluble polymer and at least one water-soluble polymer, whereas the water-soluble polymer serves as a template, for the water-insoluble polymer, which is removed by washing. As polyvinyl alcohol, polyvinyl amine, polyethylene oxide, polyvinylpyrrolidone or hydroxypropylcellulose are water-soluble polymers the resulting fibers are also water soluble and as mentioned above could be easily dissolved by washing.

WO 2008/049397 discloses the electrospinning using aqueous solution including polyelectrolytes of opposite charge.

But none of these fibers disclosed provides fluid-absorption and retention properties.

A suitable electrospinning apparatus comprises a syringe which is provided at its tip with a capillary die connected to one pole of a voltage source and is for accommodating the polymer solution to be spun. Good results are achieved when the internal diameter of the capillary die is from 50 to 500 μm. Opposite the exit of the capillary die, at a distance of about 20 cm, preferably of about 16 cm, more preferably of about 14 cm, most preferably of about 13 cm, is arranged a counterelectrode, e.g. a square counterelectrode, or collecting electrode connected to the other pole of the voltage source, which functions as the collector for the fibers formed. During the operation of the apparatus, a voltage between 0 kV and 82 kV is set at the electrodes in such a way that an electrical field of preferably from 1 to 6 kV/cm, more preferably from 1.5 to 5 kV/cm, preferentially from 2 to 4.5 kV/cm and most preferably from 2.5 to 4 kV/cm forms between the electrodes. Owing to the electrostatic charge a material flow directed toward the counterelectrode forms, which solidifies on the way to the counterelectrode with fiber formation, as a consequence of which fibers with diameters in the micro- and nanometer range are deposited on the counterelectrode.

The distance between the cannula and the counterelectrode functioning as the collector, and the voltage between the electrodes, is preferably adjusted in such a way that an electrical field of 1 to 6 kV/cm, preferably from 1.5 to 5 kV/cm, more preferably from 2 to 4.5 kV/cm and most preferably from 2.5 to 4 kV/cm forms between the electrodes. Good results are achieved especially when the internal diameter of the cannula is from 50 to 500 μm.

Also suitable are electrospinning apparatus' e.g. Nanospider® which do not use jets or capillaries for the production of fibres, but a rotating drum partially dipped in the polymer solution to be spun. The drum is rotated so that a thin film of polymer is created on its surface. Opposite to the drum, at a distance of about 20 cm, preferably of about 16 cm, more preferably of about 14 cm, most preferably of about 11 cm, a counterelectrode is arranged, e.g. a square counterelectrode, or collecting electrode connected to the other pole of the voltage source, which functions as the collector for the fibers formed.

An electrical field of preferably from 1 to 6 kV/cm, more preferably from 1.5 to 5 kV/cm, preferentially from 2 to 4.5 kV/cm and most preferably from 2.5 to 4 kV/cm is formed between the electrodes. Based on the electric field at the center of the rotating drum, which is nearest to the counterelectrode many focal points of Taylor cones are formed, which subsequently leads to a flow of matter towards the counterelectrode, the fiber spinning.

Generally a temperature range for spinning is chosen of 10 to 30° C., preferably 15 to 25° C., more preferably 21 to 27° C. and a humidity range of 10 to 45% RH, preferably 20 to 35% RH.

With the aforementioned apparatus, in accordance with the invention, a solution of at least one essentially water-soluble polymer and of at least one crosslinker in an aqueous medium is electrospun.

The acidic water-soluble polymer preferably is selected from a group comprising polyacrylic acid, hydrolyzed starchacrylonitrile graft copolymers, starch-acrylic acid graft copolymers, saponified vinyl acetate-acrylic esters copolymers, hydrolyzed acrylonitrile copolymers, hydrolyzed acrylamide copolymers, ethylene-maleic anhydride copolymers, isobutylene-maleic anhydride copolymers, poly(vinylsulfonic acid), poly(vinylphosphonic acid), poly (vinylphosphoric acid), poly(vinylsulfuric acid), sulfonated polystyrene, poly(aspartic acid), poly(lactic acid), and mixtures thereof.

An acidic water-absorbing fiber present in the inventive water-absorbing material can be either a strong or a weak acidic water-absorbing fiber. The acidic fiber can be a homopolymer or a copolymer. The identity of the acidic water-absorbing fiber is not limited as long as the fiber is capable of swelling and absorbing at least ten times its weight in water, when in a neutralized form. The acidic fiber is present in its acidic form, i.e., about 75% to 100% of the acidic moieties are present in the free acid form.

The acidic water-absorbing fiber typically is a lightly crosslinked acrylic-type fiber. The lightly crosslinked acidic fiber typically is prepared by polymerizing an acidic monomer containing an acyl moiety, e.g., acrylic acid, or a moiety capable of providing an acid group, i.e., acrylonitrile, in the presence of a crosslinker, i.e., a polyfunctional organic compound. The acidic fiber can contain other copolymerizable units, i.e., other monoethylenically unsaturated comonomers, well known in the art, as long as the polymer is substantially, i.e., at least 10%, and preferably at least 25%, acidic monomer units. To achieve the full advantage of the present invention, the acidic fiber contains at least 50%, and more preferably, at least 75%, and up to 100%, acidic monomer units. The other copolymerizable units can, for example, help improve the hydrophilicity and crosslinking of the polymer. Ethylenically unsaturated carboxylic acid and carboxylic acid anhydride monomers useful in the acidic water absorbing fibre include acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, itaconic acid, citraconic acid, mesaconic acid, glutaconic acid, aconitic acid, maleic acid, fumaric acid, tricarboxyethylene, and maleic anhydride.

Ethylenically unsaturated sulfonic acid monomers include aliphatic or aromatic vinyl sulfonic acids, such as vinylsulfonic acid, allyl sulfonic acid, vinyl toluene sulfonic acid, styrene sulfonic acid, acrylic and methacrylic sulfonic acids, such as sulfoethyl acrylate, sulfoethyl methacrylate, sulfopropyl acrylate, sulfopropyl methacrylate, 2-hydroxy-3-methacryloxypropyl sulfonic acid, and 2-acrylamide-2-methylpropane sulfonic acid.

As set forth above, polymerization of acidic monomers, and copolymerizable monomers, if present, most commonly is performed by free radical processes.

The acidic fiber, either strongly acidic or weakly acidic, can be any electrospun fiber that acts as an SAP in its neutralized form. The acidic fiber typically contains a plurality of carboxylic acid, sulfonic acid, phosphonic acid, phosphoric acid, and/or sulfuric acid moieties. Examples of acidic fibers include, but are not limited to, polyacrylic acid, hydrolyzed starchacrylonitrile graft copolymers, starch-acrylic acid graft copolymers, saponified vinyl acetate-acrylic ester copolymers, hydrolyzed acrylonitrile copolymers, hydrolyzed acrylamide copolymers, ethylene-maleic anhydride copolymers, isobutylene-maleic anhydride copolymers, poly(vinylsulfonic acid), poly(vinylphosphonic acid), poly (vinylphosphoric acid), poly(vinylsulfuric acid), sulfonated polystyrene, poly(aspartic acid), poly(lactic acid), and mixtures thereof. The preferred acidic resins are the polyacrylic acids. The water-absorbent material according to the present invention can (a) contain single kind of acidic fiber or (b) contain more than one, i.e., a mixture, of acidic fibers.

The acidic fibers are crosslinked to a sufficient extent such that the polymer is water insoluble. An acidic water-absorbing fiber can be crosslinked by suspending or dissolving a di- or polyfunctional compound capable of crosslinking the acidic fiber in the polymer solution prior to electrospinning, or the crosslinker solution may be sprayed on the fibers after electrospinning. The crosslinkers are activated by the right stimulus as e.g. heating for thermal sensitive crosslinker, or by UV light, pH-changes, redox or others.

Conventionally, the crosslinking agent is water or alcohol soluble, and possesses sufficient reactivity with the polymers of the electrospun fiber such that crosslinking occurs in a controlled fashion,

Crosslinking renders the fibers substantially water insoluble, and, in part, serves to determine the absorbent capacity of the fibers. For use in absorption applications, a fiber is lightly crosslinked, i.e., the amount of crosslinker is less than about 7% by weight, preferably the amount of crosslinker is from 0.05 to 5.0% by weight, more preferably from 0.1 to 1% by weight, most preferably from 0.3 to 0.6% by weight, based in each case on the total weight of (non-neutralized) polymer. The amount of crosslinker in % by weight is the quotient of the weight of crosslinker used and the weight of non-neutralized polymer.

Specific crosslinking monomers include, but are not limited to, 1,4-butanediol diacrylate, 1,4-butanediol dimethacrylate, 1,3-butylene glycol diacrylate, 1,3-butylene glycol dimethacrylate, diethylene glycol diacrylate, diethylene glycol dimethacrylate, ethoxylated bisphenol A diacrylate, ethoxylated bisphenolA dimethacrylate, ethylene glycol dimethacrylate, 1,6-hexanediol diacrylate, 1,6-hexanediol dimethacrylate, neopentyl glycol dimethacrylate, polyethylene glycol diacrylate, polyethylene glycol dimethacrylate, triethylene glycol diacrylate, triethylene glycol dimethacrylate, tripropylene glycoldiacrylate, tetraethylene glycol diacrylate, tetraethylene glycol dimethacrylate, dipentaerythritol pentaacrylate, pen-taerythritol tetraacrylate, pentaerythritol triacrylate, trimethylolpropane triacrylate, trimethylolpropane trimethacrylate, tris(2-hydroxyethyl)isocyanurate triacrylate, tris(2-hydroxyethyl)isocyanurate trimethacrylate, divinyl esters of a polycarboxylic acid, diallyl esters of a polycarboxylic acid, triallyl terephthalate, diallyl maleate, diallyl fumarate, hexamethylenebismaleimide, trivinyl trimellitate, divinyl adipate, diallyl succinate, a divinyl ether of ethylene glycol, cyclopentadiene diacrylate, tetra allyl ammonium halides, or mixtures thereof. Compounds such as divinylbenzene and divinyl ether also can be used to crosslink the poly(dialkylaminoalkyl acrylamides). Especially preferred crosslinking agents are N,N′-methylenebisacrylamide, N,N′-methylenebismethacrylamide, ethylene glycol dimethacrylate, and trimethylolpropane triacrylate.

Furthermore an acidic water-absorbing fiber can also be crosslinked e.g. by suspending or dissolving a di- or polyfunctional compound capable of crosslinking the acidic fiber by reaction with the acid-groups of the acid polymer in the polymer solution to be electrospun.

The basic water-soluble polymer preferably is selected from a group of comprising gpoly(vinylamine), poly(dialkylaminoalkyl(meth)acrylamide), polymer prepared from the ester analog of N-(dialkylamido(meth)acrylamide), a polyethyleneiminen a poly(vinylgunidine), a poly(allylguanidine), a poly(allylamine), poly(dimethyldialkylammonium hydroxide), guanidinemodified polysterene, quternized polystyrene, quaternized poly(meth)acrylamide or ester analog thereof, poly(vinal alcohol-co-vinylamine), and mixtures thereof.

The crosslinking can be performed by polyfunctional organic compounds, as set forth above with respect to the acidic water-absorbing fiber.

Conventionally, the crosslinking agent is water or alcohol soluble, and possesses sufficient reactivity with the polymers of the electrospun fiber such that crosslinking occurs in a controlled fashion,

A basic water-absorbing fiber typically contains an amino or a guanidino group.

Accordingly, a water-soluble basic fiber also can be crosslinked by suspending or dissolving a di- or polyfunctional compound capable of crosslinking the basic fiber by reaction with the amino groups of the basic polymer in the polymer solution to be electrospun.

Such crosslinking agents include, for example, multifunctional aldehydes (e.g., glutaraldehyde), multifunctional acrylates (e.g., butanediol diacrylate, TMPTA), halohydrins (e.g., epichlorohydrin),dihalides (e.g., dibromopropane), disulfonate esters (e.g., ZA(0₂)O—(CH₂)n-OS(O)₂Z, wherein n is 1 to 10, and Z is -methyl or -tosyl), multifunctional epoxies (e.g., ethyleneglycol diglycidyl ether), multifunctional esters (e.g., dimethyladipate), multifunctional acid halides (e.g., oxalyls chloride), multifunctional carboxylic acids (e.g., succinicacid), carboxylic acid anhydrides (e.g., succinic anhydride),organic titanates (e.g., TYZOR AA from DuPont), melamine resins (e.g., CYMEL 301, CYMEL 303, CYMEL 370, and CYMEL 373 from Cytec Industries, Wayne, N.J.), hydroxymethyl ureas (e.g., N,N′-dihydroxymethyl-4,5-dihydroxyethyleneurea), and multifunctional isocyanates(e.g., toluene diisocyanate or methylene diisocyanate).

Crosslinking agents also are disclosed in U.S. Pat. No. 5,085,787, incorporated herein by reference, and in EP 450 923.

The basic fiber, either strongly or weakly basic, therefore, can be any electrospun fiber that acts as a water absorbing material in its charged form. The water-absorbent material according to the present invention can (a) contain single kind of basic fiber or (b) contain more than one, i.e., a mixture, of basic fibers.

Analogous to the acidic fiber, the basic water-absorbing fiber in the present SAP fibers can be a strong or weak basic water-absorbing fiber. The basic fiber can be a homopolymer or a copolymer.

The identity of the basic fiber is not limited as long as the basic fiber is capable of swelling and absorbing at least 10 times its weight in water, when in a charged form. The weak basic fiber typically is present in its free base, or neutral, form, i.e., about 75% to about 100% of the basic moieties, e.g., amino groups, are present in a neutral, uncharged form. The strong basic fiber typically are present in the hydroxide (OH) or bicarbonate (HC0₃) form.

The polymer solutions have typically been to 0% to 25% neutralized. Neutralization is preferably carried out at the monomer stage. This is typically done by mixing in the neutralizing agent as an aqueous solution or preferably also as a solid. The degree of neutralization is preferably from 0 to 25 mol %, more preferably from 5 to 15 mol %, most preferably from 6 to 10 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts, such as the salt of triethanolamine. Particularly preferred alkali metals are sodium and potassium, but very particular preference is given to sodium hydroxide, sodium carbonate or sodium hydrogencarbonate and also mixtures thereof.

For the electro-spinning process it is important that the polymer concentration in the aqueous medium is between 5% and 60%, preferably between 8% and 50%, more preferably between 10 to 30%.

When exclusively water is used as the solvent, a surfactant may be advantageously added. This improve the electrospinning process and the fiber properties. Suitable sufactants are surfactants comprising e.g. (oligo)oxyalkene groups, or carbohydrate groups or amine oxides.

The fibers are collected by a substrate sheet on the collector, such as a nonwoven sheet, e.g. of polypropylene, or an aluminium foil sheet, or substrates of silica treated paper, metal or other suitable material. The substrate is necessary for anti-charging the fibres and to avoid distribution of the fibers throughout the chamber, where the spinning is performed, which will cause disruption of the spinning process.

Preferably the electrospun fibres (either acidic or basic) are collected on a substrate until a layer of the fibres had been formed. Different layers are for example combined by electrospinning different polymer solutions one after another and collecting the fibres produced out of each polymer solution on the same substrate until a layer is formed in each case.

According to the invention the layers could be either acidic or basic, whereas they could be arranged in every useful pattern.

Whereas the fiber spinning situations, such as polymer concentration in the solution to be spun for each polymer, types of crosslinking agents and crosslinking concentration may influence the respective layers and the pattern.

The weight of each layer may be determined by weighing the substrate with the layer and subtracting the weight of the substrate or the substrate with the previous spun layers respectively.

The crosslinker in the polymer fibres can be activated after each spinning process, this means after each solution of water-soluble polymers to be electrospun and collected on a substrate or it may be preferable to collect two or more layers of electrospun fibres generated of the same or different sulutions of water-soluble polymers on the same substrate and then crosslinking the fibres.

It is preferred to collect at least 2 layers, more preferably at least 3 layers, most preferably at least 5 layers of fibres and crosslink them together at the same time.

The electrospun fibers are crosslinked by activating the crosslinking agent after electrospinning e.g. by heating for thermal sensitive crosslinking agents, or by UV light, pH-changes or redox, for other crosslinking agents after the spinning process. If using thermal sensitive crosslinking agents the fibres after electrospinning are heated to a temperature of 60 to 220° C., preferably 90 to 200° C., more preferably 110 to 180° C., most preferably 120 to 140° C., preferentially 125 to 135. Whereas the heating is performed for at least 10 min, preferably 30 to 90 min, more preferably 45 to 75 min, most preferably 55 to 65 min.

The heating may be performed without any extra drying step as an extra drying step is not necessary because of the diameter in the micro- or nanometer range and the very large surface area of the fibers.

To activate the heat sensitive crosslinking agent the heating could be performed by different methods one suitable method is to place the fibers on a tray, e.g. a metal sheet or any other suitable sheet and put this tray into an oven at a temperature and a time period suitable for activating the respective crosslinking agent as. Afterwards the fibers were removed from the substrate.

In case the substrate is resistant to the heat applied the fibers are not removed from the substrate and the substrate with the fibres is put into the oven.

After the heating process, the moisture content in fibers is preferably 0.5 to 15% by weight, more preferably 1 to 10% by weight, most preferably 3 to 5% by weight, the residual moisture content being determined by EDANA recommended test method No. WSP 230.2-5 “Moisture Content”.

In the case of too high a residual moisture content, the dried polymer fibers have too low a glass transition temperature T_{g} and can be processed further only with difficulty. In the case of too low a residual moisture content, the dried polymer gel is too brittle and, in the subsequent comminution steps, undesirably large amounts of polymer fibers with an excessively low fiber length are obtained.

It is preferred to cool the polymer fibers after thermal activation. The cooling is preferably carried out in coolers (moisture controlled ovens, set at room temperature) at a temperature range from 18 to 25° C.

In a further embodiment of the present invention no crosslinker is added to the polymer solution to be electrospun. The resulting fibers which are water soluble are crosslinked by exposing the fibers to UV-light. Wherein the UV radiation has a wavelength of 50 to 350 nm, preferably 100 to 300 nm.

The degree of crosslinking could be determined e.g. by the time and especially the intensity of the UV radiation.

The water-absorbing fibers combine a high water resistance with a good mechanical stability. Furthermore the SAP fibers according to the present invention absorb liquids quickly, provide a good fluid permeability and conductivity into and through the SAP fiber and have high gel strength such that the hydrogel formed from the SAP fibers does not deform or flow under an applied pressure or stress.

The diameter of the inventive fibers is preferably less than 3 μm, preferably less than 2 μm, more preferably less than 1 μm, particularly less than 500 nm, very particularly less than 300 nm. The length of the fibers depends upon the intended use and is generally from 50 μm up to several kilometres.

To further improve the properties, the polymer fibers can be coated and/or remoisturized.

Suitable coatings for controlling the acquisition behavior and improving the permeability (SFC or GBP) are, for example, inorganic inert substances, such as water-insoluble metal salts, organic polymers, cationic polymers and polyvalent metal cations. Suitable coatings for improving the color stability are, for example reducing agents and anti-oxidants. Suitable coatings for dust binding are, for example, polyols.

Suitable inorganic inert substances are silicates such as montmorillonite, kaolinite and talc, zeolites, activated carbons, polysilicic acids, magnesium carbonate, calcium carbonate, calcium phosphate, barium sulfate, aluminum oxide, titanium dioxide and iron(II) oxide. Preference is given to using polysilicic acids, which are divided between precipitated silicas and fumed silicas according to their mode of preparation. The two variants are commercially available under the names Silica FK, Sipernat®, Wessalon® (precipitated silicas) and Aerosil® (fumed silicas) respectively. The inorganic inert substances may be used as dispersion in an aqueous or water-miscible dispersant or in substance.

When the fluid-absorbent polymer fibers are coated with inorganic inert substances, the amount of inorganic inert substances used, based on the fluid-absorbent polymer fibers, is preferably from 0.05 to 5% by weight, more preferably from 0.1 to 1.5% by weight, most preferably from 0.3 to 1% by weight.

Suitable organic polymers are polyalkyl methacrylates or thermoplastics such as polyvinyl chloride, waxes based on polyethylene, polypropylene, polyamides or polytetrafluoro-ethylene. Other examples are styrene-isoprene-styrene block-copolymers or styrene-butadiene-styrene block-copolymers.

Suitable cationic polymers are polyalkylenepolyamines, cationic derivatives of polyacrylamides, polyethyleneimines and polyquaternary amines.

Polyquaternary amines are, for example, condensation products of hexamethylenedi-amine, dimethylamine and epichlorohydrin, condensation products of dimethylamine and epichlorohydrin, copolymers of hydroxyethylcellulose and diallyldimethylammo-nium chloride, copolymers of acrylamide and α-methacryloyloxyethyltrimethylammo-nium chloride, condensation products of hydroxyethylcellulose, epichlorohydrin and trimethylamine, homopolymers of diallyldimethylammonium chloride and addition products of epichlorohydrin to amidoamines. In addition, polyquaternary amines can be obtained by reacting dimethyl sulfate with polymers such as polyethyleneimines, copolymers of vinylpyrrolidone and dimethylaminoethyl methacrylate or copolymers of ethyl methacrylate and diethylaminoethyl methacrylate. The polyquaternary amines are available within a wide molecular weight range.

However, it is also possible to generate the cationic polymers on the fiber surface, either through reagents which can form a network with themselves, such as addition products of epichlorohydrin to polyamidoamines, or through the application of cationic polymers which can react with an added crosslinker, such as polyamines or polyimines in combination with polyepoxides, polyfunctional esters, polyfunctional acids or poly-functional (meth)acrylates.

It is possible to use all polyfunctional amines having primary or secondary amino groups, such as polyethyleneimine, polyallylamine and polylysine. The liquid sprayed by the process according to the invention preferably comprises at least one polyamine, for example polyvinylamine or a partially hydrolyzed polyvinylformamide.

The cationic polymers may be used as a solution in an aqueous or water-miscible solvent, as dispersion in an aqueous or water-miscible dispersant or in substance.

When the fluid-absorbent polymer fibers are coated with a cationic polymer, the use amount of cationic polymer based on the fluid-absorbent polymer particles is usually not less than 0.001% by weight, typically not less than 0.01% by weight, preferably from 0.1 to 15% by weight, more preferably from 0.5 to 10% by weight, most preferably from 1 to 5% by weight.

Suitable polyvalent metal cations are Mg²⁺, Ca²⁺, Al³⁺, Sc³⁺, Ti⁴⁺, Mn²⁺, Fe^{2+/3+}, Co²⁺, Ni²⁺, Cu+^{+/2+}, Zn²⁺, Y³⁺, Zr⁴⁺, Ag+, La³⁺, Ce⁴⁺, Hf⁴⁺ and Au^{+/3+}; preferred metal cations are Mg²⁺, Ca²⁺, Al³⁺, Ti⁴⁺, Zr⁴⁺ and La³⁺; particularly preferred metal cations are Al³⁺, Ti⁴⁺ and Zr⁴⁺. The metal cations may be used either alone or in a mixture with one another. Suitable metal salts of the metal cations mentioned are all of those which have a sufficient solubility in the solvent to be used.

Particularly suitable metal salts have weakly complexing anions, such as chloride, hydroxide, carbonate, nitrate and sulfate. The metal salts are preferably used as a solution or as a stable aqueous colloidal dispersion. The solvents used for the metal salts may be water, alcohols, dimethylfor-mamide, dimethyl sulfoxide and mixtures thereof. Particular preference is given to water and water/alcohol mixtures, such as water/methanol, water/isopropanol, water/1,3-propanediole, water/1,2-propandiole/1,4-butanediole or water/propylene glycol.

When the fluid-absorbent polymer fibers are coated with a polyvalent metal cation, the amount of polyvalent metal cation used, based on the fluid-absorbent polymer particles, is preferably from 0.05 to 5% by weight, more preferably from 0.1 to 1.5% by weight, most preferably from 0.3 to 1% by weight.

Suitable reducing agents are, for example, sodium sulfite, sodium hydrogensulfite (sodium bisulfite), sodium dithionite, sulfinic acids and salts thereof, ascorbic acid, sodium hypophosphite, sodium phosphite, and phosphinic acids and salts thereof. Preference is given, however, to salts of hypophosphorous acid, for example sodium hypophos-phite, salts of sulfinic acids, for example the disodium salt of 2-hydroxy-2-sulfinato-acetic acid, and addition products of aldehydes, for example the disodium salt of 2-hy-droxy-2-sulfonatoacetic acid. The reducing agent used can be, however, a mixture of the sodium salt of 2-hydroxy-2-sulfinatoacetic acid, the disodium salt of 2-hydroxy-2-sulfonatoacetic acid and sodium bisulfite. Such mixtures are obtainable as Bruggolite® FF6 and Bruggolite® FF7 (Bruggemann Chemicals; Heilbronn; Germany).

The reducing agents are typically used in the form of a solution in a suitable solvent, preferably water. The reducing agent may be used as a pure substance or any mixture of the above reducing agents may be used.

When the fluid-absorbent polymer fibers are coated with a reducing agent, the amount of reducing agent used, based on the fluid-absorbent polymer particles, is preferably from 0.01 to 5% by weight, more preferably from 0.05 to 2% by weight, most preferably from 0.1 to 1% by weight.

Suitable polyols are polyethylene glycols having a molecular weight of from 400 to 20000 g/mol, polyglycerol, 3- to 100-tuply ethoxylated polyols, such as trimethylol-propane, glycerol, sorbitol and neopentyl glycol. Particularly suitable polyols are 7- to 20-tuply ethoxylated glycerol or trimethylolpropane, for example Polyol TP 70® (Perstorp AB, Perstorp, Sweden). The latter have the advantage in particular that they lower the surface tension of an aqueous extract of the fluid-absorbent polymer fibers only insignificantly. The polyols are preferably used as a solution in aqueous or water-miscible solvents.

When the fluid-absorbent polymer fibers are coated with a polyol, the use amount of polyol, based on the fluid-absorbent polymer particles, is preferably from 0.005 to 2% by weight, more preferably from 0.01 to 1% by weight, most preferably from 0.05 to 0.5% by weight.

The coating is preferably performed by spaying the respective solutions on the fibers or on the fiber layers.

The inventive absorbing material may be useful in hygiene products, as filter material for water-desalination or may be used for condensation prevention in e.g. LED-lights.

Especially the present invention is also related to fluid absorbent articles comprising water-absorbing material according to the invention. Fluid absorbent articles are understood to mean, for example, incontinence pads and incontinence pants for adults, or diapers for babies.

Furthermore the invention is directed to a fluid absorbent article comprising a sheet containing water-absorbing material according to the present invention. The sheet preferably contains water-absorbing material in an amount of about 90 to 100% by total weight of the electrospun fibres.

For example, the resulting fluid absorbent article may have the following construction:

(A) an upper liquid-pervious topsheet
(B) a lower liquid-impervious layer
(C) a sheet comprising water-absorbing material with or without fluff, between topsheet (A) and layer (B),
(D) optionally a tissue layer immediately above and below sheet (C) and
(E) optionally an absorption and distribution layer between topsheet (A) and the s (C).

The thickness of sheet (C), especially of the water absorbing material or the water absorbing material with fluff can be varied. For example, sheet (C) may have less material, for example, in the outer region. Cutouts and channels are likewise possible.

Examples of fibers to be mixed with the inventive water absorbing material include cellulose fibers such as fluff pulp and cellulose of the cotton type. The materials (soft- or hardwoods), production processes such as chemical pulp, semichemical pulp, chemothermomechanical pulp (CTMP) and bleaching processes are not particularly restricted. For example, natural cellulose fibers such as cotton, flax, silk, wool, jute, ethylcellulose and cellulose acetate are used.

Suitable synthetic fibers are produced from polyvinyl chloride, polyvinyl fluoride, polytetrafluoro-ethylene, polyvinylidene chloride, polyacrylic compounds such as ORLON®, polyvinyl acetate, polyethyl vinyl acetate, soluble or insoluble polyvinyl alcohol. Examples of synthetic fibers include thermoplastic polyolefin fibers, such as polyethylene fibers (PULPEX®), polypropylene fibers and polyethylene-polypropylene bicomponent fibers, polyester fibers, such as polyethylene terephthalate fibers (DACRON® or KODEL®), copolyesters, polyvinyl acetate, polyethyl vinyl acetate, polyvinyl chloride, polyvinylidene chloride, polyacrylics, polyamides, copolyamides, polystyrene and copolymers of the aforementioned polymers and also bicomponent fibers composed of polyethylene terephthalate-polyethylene-isophthalate copolymer, polyethyl vinyl acetate/polypropylene, polyethylene/polyester, polypropylene/polyester, copolyester/polyester, polyamide fibers (nylon), polyurethane fibers, polystyrene fibers and polyacrylonitrile fibers. Preference is given to polyolefin fibers, polyester fibers and their bicomponent fibers. Preference is further given to thermally adhesive bicomponent fibers composed of polyolefin of the core-sheath type and side-by-side type on account of their excellent dimensional stability following fluid absorption.

The fiber cross section may be round or angular, or else have another shape, for example like that of a butterfly.

The synthetic fibers mentioned are preferably used in combination with thermoplastic fibers. In the course of the heat treatment, the latter migrate to some extent into the matrix of the fiber material present and so constitute bond sites and renewed stiffening elements on cooling. In addition, the addition of thermoplastic fibers means that there is an increase in the present pore dimensions after the heat treatment has taken place. This makes it possible, by continuous metered addition of thermoplastic fibers during the formation of the absorbent layer, to continuously increase the fraction of thermoplastic fibers in the direction of the topsheet, which results in a similarly continuous increase in the pore sizes. Thermoplastic fibers can be formed from a multitude of thermoplastic polymers which have a melting point of less than 190° C., preferably in the range from 75° C. to 175° C. These temperatures are too low for damage to the cellulose fibers to be likely.

Lengths and diameters of the above-described synthetic fibers are not particularly restricted, and generally any fiber from 1 to 200 mm in length and from 0.1 to 100 denier (gram per 9000 meters) in diameter may preferably be used. Preferred thermoplastic fibers are from 3 to 50 mm in length, particularly preferred thermoplastic fibers are from 6 to 12 mm in length. The preferred diameter for the thermoplastic fibers is in the range from 1.4 to 10 decitex, and the range from 1.7 to 3.3 decitex (gram per 10 000 meters) is particularly preferred. The form of the fibers may vary; examples include woven types, narrow cylindrical types, cut/split yarn types, staple fiber types and continuous filament fiber types.

Suitable hydrophilic fibers include for example cellulose fibers, modified cellulose fibers, rayon, polyester fibers, for example polyethylene terephthalate (DACRON®), and hydrophilic nylon (HYDROFIL®). Suitable hydrophilic fibers may also be obtained by hydrophilizing hydrophobic fibers, for example the treatment of thermoplastic fibers obtained from polyolefins (e.g. polyethylene or polypropylene, polyamides, polystyrenes, polyurethanes, etc.) with surfactants or silica. However, for reasons of cost and availability, cellulose fibers are preferred.

The liquid-impervious topsheet (A) is a layer in direct contact with the skin. The material for this purpose consists of customary synthetic or semisynthetic fibers or films of polyester, polyolefins, rayon or natural fibers such as cotton. In the case of nonwoven materials, the fibers should generally be bound by binders such as polyacrylates. Preferred materials are polyester, rayon and blends thereof, polyethylene and polypropylene. Examples of liquid-pervious layers are described in WO 99/57355 A1, EP 1 023 883 A2.

The liquid-impervious layer (B) generally consists of a film of polyethylene or polypropylene. A nonwoven may be laminated onto the layer (B) for better tactile properties on the outside.

Absorption and distribution layers (E) are typically produced from nonwovens which have very good wicking action, in order to absorb and to distribute the liquid rapidly. They also improve rewetting. When pressure on the diaper causes the water-absorbing composite to release liquid, the absorption and distribution layer (E) prevents this liquid from coming into contact with the skin of the user.

Suitable nonwovens are thermally bonded or resin-bonded fibers based on polypropylene and/or polyester fibers with a basis weight of 25 to 70 gms, for example Curadis@, Curadis® EPS, Curadis® ATP and Curadis® RB (Albis SPA, IT).

Further suitable absorption and distribution layers (E) are obtained by “airthroughbonding” and are obtainable under the Acquitex® (Texus SPA, IT) and Dry Web® (Libeltex BVBA, NL) trademarks.

### Methods

### Solid Content

The solids content within the solution is determined gravimetrically by means of a Mettler Toledo HR73 halogen moisture analyzer, by heating approx. 1 ml of the sample to 200° C. within 2 minutes and drying the sample to constant weight and then weighing it.

### Size of the Fibers

The size, i.e. the diameter and the length of the fibers, is determined by evaluating electron micrographs (using a Scanning Electronic Microscope (SEM) to determine the fiber diameters).

### Absorbent Capacity

The Absorbent Capacity of fluid-absorbent material (SAP) is determined by the following method:
In saline solution:
0.010 g+/−0.001 g of SAP are carefully put into a 140 micron, water-permeable mesh attached to the base of a hollow plexiglass cylinder with an internal diameter of 25 mm. The SAP is covered with a 4.400 g+/−0.050 g cover plate. Then the weight of the cylinder assembly is measured. After weighing the screened base of the cylinder is placed in a 10 ml petri dish or a weight dish containing 3.5 g of a 0.9% by weight saline solution, whereas the SAP/saline solution ratio is 1/35. The polymer is allowed to absorb the saline solution for 1 hour. Then, using vacuum equipment (generating a vacuum of about 82.7 kPa) for about 10 seconds to withdraw the interstitial fluid. Then, by reweighing the cylinder assembly, the Absorbent Capacity is calculated by the ratio of the weight of liquid absorbed over the dry weight of polymer before liquid contact.
In deionized water:
The Absorbent Capacity is also determined in deionized water. Here the amount of saline solution of the method described above is replaced by the same or a higher amount of deionized water.
For SAP particles (e.g. T 8760) a 30 ml petri dish was used instead of a 10 ml petri dish.

### Moisture Content

The moisture content of the fluid-absorbent polymer particles is determined by the EDANA recommended test method No. WSP 230.2-5 “Moisture Content”.

### Residual Monomers

The level of residual monomers in the fluid-absorbent polymer particles is determined by the EDANA recommended test method No. WSP 210.2-5 “Residual Monomers”.

The EDANA test methods are obtainable, for example, from the EDANA, Avenue Eugene Plasky 157, B-1030 Brussels, Belgium.

### Desalination Index

The Desalination Index is calculated for each SAP by the ratio of the Absorbent capacity measured for this SAP in saline over the Absorbent capacity measured for the SAP in de-ionized water. (Desalination Index (DI)=(Absorbent capacity for SAP in saline)/(Absorbent capacity for SAP in deionized water)

### EXAMPLES

The following examples illustrate the preparation of the SAP-fibers of the present invention.

For the examples a commercial available poly-acrylic acid (PAA) named Sokalan® PA 110S (about 35% of polymer, available from, BASF SE, Carl-Bosch-Strasse 38, 6703, Ludwigshafen, Germany) is diluted to a suitable concentration with de-ionized water, and the resulting solution is neutralized to designed degree levels. Further a selected crosslinking agent is dissolved into the solution. This preparation should unless stated otherwise, be carried out at an ambient temperature of 23±2° C. and an atmospheric humidity of 50±10%.

### Preparation of Polyvinylamine

All given percentages are weight % if not mentioned otherwise.

Solid contents were measured in a forced draft oven: 2 h at 140° C.

K-values are determined according to H. Fikentscher, Cellulosechemie, volume 13 pages 48-64 and 71-74 (1932). The specific conditions are mentioned in brackets

Molecular Weight is determined via static light scattering in an aqueous 0.6% NaCl solution at a pH of 7.6

The degree of hydrolysis is determined by measuring the formed formate. For this purpose an enzymatic test set for formic acid of R-Biopharm AG, Darmstadt, Germany is used

### Preparation of Polyvinylamine GK 2214/131

A 3 necked 4 litre glass vessel equipped with anchor stirrer, condenser, thermo couple and nitrogen inlet is charged with 1931.4 g of distilled water and 7.7 g of 75% phosphoric acid. While stirring at 100 rpm 10.1 g of 25% caustic soda is added to achieve a pH of 6.5. While bubbling nitrogen through the solution for 30 min the vessel is heated to 78° C. Vacuum is applied to such a level (about 480 mbar) that the buffer solution starts boiling without distilling off a significant amount. 759.1 g of N-vinylformamide and a solution of 13.5 g of 2,2″-Azobis(2-methylpropionamide)-dihydrochloride in 135 g of distilled water is added in 3 hours simultaneously. After about 20 min from the start of the two feeds polymerisation become visible by an increased distillation rate. During the polymerization water is distilled off to remove the polymerization heat. During the 3 hours temperature is kept constant at 78° C. by adjusting the vacuum accordingly. After the end of the 2 feeds the internal temperature is kept at 78° C. for another 2 h. During the whole polymerisation process 783 g of water are distilled off. Vacuum is released by venting with nitrogen and a sample is taken to determine the analytical data. A viscous lightly yellow solution of Polyvinylformamide (PVFA) is obtained.

| | |
|---|---|
| K-value (1% in water): | 49 |
| Mw | 48 000 |
| Solid content: | 36.6% |

The achieved polymer solution is heated to 80° C. To hydrolyse the PVFA 55.1 g of a 40% aqueous solution of sodium-hydrogensulphite and 1863.7 g of 25% aqueous caustic soda is added. The reaction mixture is held for 6 h at 80° C. After cooling to room temperature an aqueous solution of polyvinylamine (PVAm) with a degree of hydrolysis of 98 mol % is obtained.

3937 g of the PVAm solution is diluted with 2384 g distilled water and heated to 50° C. To remove the formate formed during hydrolysis the solution is ultra-filtrated using a MPS-34 membrane of Kiryat Weizmann LTD, Rehovot, Israel with a cut of limit of 500 Dalton. In total 26545 g of filtrate are replaced by distilled water. By this means >99.5% of the formate is removed. The final product has a solid content 8.6%

### Preparation of Polyvinylamine GK 2873/035

A 3 necked 4 litre glass vessel equipped with anchor stirrer, condenser, thermo couple and nitrogen inlet is charged with 1235.3 g of distilled water and 2.9 g of 75% phosphoric acid. While stirring at 100 rpm 4.3 g of 25% caustic soda is added to achieve a pH of 6.5. While bubbling nitrogen through the solution for 30 min the vessel is heated to 77° C. Vacuum is applied to such a level (about 410 mbar) that the buffer solution starts boiling without distilling off a significant amount. Simultaneously a feed of 262.3 g of N-vinylformamide (VFA) and a feed of 1.2 g of 2,2′-Azobis(2-methylpropionamide)-dihydrochloride in 65.0 g of distilled water is started. The VFA feed lasts 2 h while the initiator feed is added in 2 h 50 min. After about 20 min from the start of the two feeds polymerization becomes visible by an increased distillation rate. During the polymerisation water is distilled off to remove the polymerization heat. During the feeding period temperature is kept constant at 77° C. by adjusting the vacuum accordingly. After the end of the initiator feed the internal temperature is kept at 77° C. for another 3 h. During the whole polymerisation process 260 g of water was distilled off. Vacuum is released by venting with nitrogen and 708.5 g distilled water is added. A sample is taken to determine the analytical data. A viscous lightly yellow solution of polyvinylformamide (PVFA) is obtained.

| | |
|---|---|
| K-value (1% in water): | 89 |
| Mw | 340 000 |
| Solid content: | 13.0% |

The achieved polymer solution is heated to 80° C. To hydrolyse the PVFA 7.1 g of a 40% aqueous solution of sodium-hydrogensulphite and 1096.1 g of 25% aqueous caustic soda is added. The reaction mixture is held for 7 h at 80° C. After cooling to room temperature an aqueous solution of polyvinylamine with a degree of hydrolysis of 99 mol % is obtained. 2958 g of the PVAm solution is diluted with 3106 g distilled water and heated to 50° C. To remove the formate formed during hydrolysis the solution is ultra-filtrated at 50° C. using a membrane of NG Technology Corporation, Needham, Mass., USA, Israel with a cut of limit of 3000 Dalton. In total 27072 g of filtrate is replaced by distilled water. By this means >99.8% of the formate was removed. The final product has a solid content of 6.5%

### PVAm Solution Used for Electrospinning

70 gram of a 4 wt % salt free Polyvinylamine (PVAm, GK 2873/035, average molecular weight is about 340,000) and 30 gram of a 4 wt % salt free Polyvinylamine (GK 2214/131, average molecular weight is about 45,000) are mixed; 0.01 gram of triton X-100 (available from Sigma-Aldrich) added and stirred about 30 minutes with a stir bar.

### Preparation of a 20% PAA Solution

A 20% PAA solution is prepared made by mixing 228.57 grams of 35% PAA (poly-acrylic acid) solution (Sokalan® PA 110S, BASF SE, Carl-Bosch-Strasse 38, 6703, Ludwigshafen, Germany) with 171.43 grams of de-ionic water by using of a stir plate with a stir bar for 30 minutes.

### Electrospinning

The electrospinning was performed with an electrospinning spider NS LAB 200, commercial available from Elmarco s.r.o. V Horkach 76/18, 460 07, Liberec 9, Czech Republic.

For each electrospinning example about 20 grams of the respective solution are poured into the Nanospider®'s low volume 20 ml spinning tube with small cylinder spinning electrode. The Tube is then placed into Nanospider® chamber with Elmarco's (anti-stable charge treated) nonwoven substrate. The cylinder in the tube is positioned 13-14 cm in a distance from the collecting electrode. Generally a temperature range for spinning is chosen to 10 to 30° C. range and humidity range of 10 to 45% RH. For the polymer used in Examples 1 to 12 the optimum conditions are a temperature range of 21 to 27° C. and humidity in the range of 20-35% RH.

Providing an electric field strength in the range of 1 to 5 kV/cm the E-spun fibers are made and collected on the selected substrates.

### Example 1 (Comparative)

50 grams of the solution of 20% PAA are mixed with 11.11 grams of Sodium Hydroxide Solution Certified 50/w/w (50/50 sodium hydroxide/water weight ratio, available from Fisher Scientific, 3970 Johns Creek Ct., Suit 500, Suwanee, Ga. 30024, USA) and stirred for 60 minutes on stir plate with a stir bar. Small air bubbles are seen throughout the solution, so the solution is sit until all of the air bubbles are dissipated. The degree of neutralization (DN) of the PAA solution is 50%.

Denacol EX-810 (Nagase ChemteX Corporation Tasuno City Hyogo, Japan) is used as a crosslinker; 0.10 grams of Denacol EX-810 are added into 50 grams of a PAA solution and stirred about 30 minutes with a stir bar.

After the electrospinning was performed, the e-spun fibers formed are kept on the substrate. The substrate is placed on a metal sheet and put into a 130° C. oven for 60 minutes to activate the crosslinker. The fibers then removed from the substrate.

### Example 2 (Comparative)

50 grams of the 20% PAA solution are mixed with 6.67 grams of Sodium Hydroxide Solution Certified 50/w/w (50/50 sodium hydroxide/water weight ratio, available from Fisher Scientific, 3970 Johns Creek Ct., Suit 500, Suwanee, Ga. 30024, USA) and stirred for 60 minutes on stir plate with a stir bar. Small air bubbles are seen throughout the solution, so the solution is sit until all of the air bubbles are dissipated. The degree of neutralization (DN) of the PAA solution is 30%.

Denacol EX-810 (Nagase ChemteX Corporation Tasuno City Hyogo, Japan) is used as a crosslinker; 0.10 grams of Denacol EX-810 are added into 50 grams of a PAA solution and stirred about 30 minutes with a stir bar.

After the electrospinning was performed, the e-spun fibers formed are kept on the substrate. The substrate is placed on a metal sheet and put into a 130° C. oven for 60 minutes to activate the crosslinker. The fibers then removed from the substrate.

### Example 3 (Comparative)

70 gram of a 4 wt % salt free Polyvinylamine (PVAm, GK 2873/035, average molecular weight is about 340,000) and 30 gram of a 4 wt % salt free Polyvinylamine (GK 2214/131, average molecular weight is about 45,000) are mixed; 0.01 gram of diluted Triton X-100 with 1 wt % of Triton concentration (available from Sigma-Aldrich) added and 0.2 gram of hydroxypropyl acrylate (available from BASF Corporation, 2090 Wagner Street, Vandalia, Ill. 62471, USA) is added and stirred about 30 minutes with a stir bar. After the electrospinning was performed, the E-spun fibers are exposed to 130° C. for 60 minutes.

### Example 4 (Comparative)

20 ml of the solution of 20% PAA are electrospun. After the electrospinning was performed, the fibres are kept on the substrate. The substrate is placed on a metal sheet and put into a 130° C. oven for 60 minutes.

### Example 5 (Comparative)

### Preparation of the MDC Particles

### Step 1: Preparation of PAA (Polyacrylic Acid) Particles

A monomer mixture containing 270 g acrylic acid, 810 g deionized water, 0.4 g methylenebisacrylamide, 0.547 g sodium persulfate, and 0.157 g 2-hydroxy-2-methyl-1-phenyl-propan-1-one was prepared, then sparged with nitrogen for 15 minutes. The monomer mixture was placed into a shallow glass dish, then the monomer mixture was polymerized under 15 mW/cm² of UV light for 25 minutes. The resulting polyacrylic acid was a rubbery gel.

The rubbery polyacrylic acid gel was cut into small pieces, then extruded through a KitchenAid Model K5SS mixer with meat grinder attachment. The extruded gel was dried in a forced-air oven at 120° C., and finally ground and sized through sieves to obtain the desired particle size.

This procedure provided a lightly crosslinked polyacrylic acid hydrogel (particles) with a degree of neutralization of zero (DN=0).

### Step 2: Preparation of PVAm (Polyvinylamine) Particles

To 2 liters of a 3% by weight aqueous polyvinylamine solution was added 0.18 g of ethyleneglycol diglycidyl ether (EGDGE). The resulting mixture was stirred to dissolve the EGDGE, then the mixture was heated to about 60° C. and held for one hour to gel. The gel was heated to about 80° C. and held until about 90% of the water was removed. The resulting gel then was extruded and dried to a constant weight at 80° C. The dried, lightly crosslinked polyvinylamine then was cryogenically milled to form a granular material (hydrogel).

### Step 3: Preparation of MDC (Multicomponent Superabsorbent) Particles

Thirty grams of the polyvinylamine granular material made by step 2 were extruded through a KitchenAid Model K5SS mixer with meat grinder attachment. Thirty grams of the polyacrylic acid hydrogel made by step 1 also were extruded through a KitchenAid Model K5SS mixer with meat grinder attachment. The two extrudates then were combined via hand mixing, followed by extruding the resulting mixture two times using the meat grinder. The extruded product then was dried for 16 hours at 60° C., milled and sized to 180-710 μm. The procedure yields multicomponent SAP containing microdomains of poly polyvinylamine and polyacrylic acid, and having polyvinylamine/polyacrylic acid weight ratio of about 50/50.

### Example 6 (Inventive)

An equal amount of the crosslinked-fibres according to examples 3 and 4 were mixed.

### Example 7 (Inventive)

20 ml of the solution of 20% PAA are electrospun. During electrospinning the fibres are collected on a substrate until a layer of the fibres had been formed. The weight of the layer is determined by weighing the substrate with the layer and subtracting the weight of the substrate. Then the electrospinning is performed with the polyvinylamine solution of example 3 and the fibres are collected on the same substrate on top of the PAA-fibres. The substrate is weighed and the weight of the PVAm fibres determined. The ratio of the fibres in total are 35% PAA and 65% PVAm. Then the substrate is placed on a metal sheet and put into a 130° C. oven for 60 minutes.

### Example 8 (Inventive)

The process is performed according to Example 7, but on top of the PVAm layer a third layer of PVAm fibres is formed. The ratio of the fibres in total are 50% PAA and 50% PVAm.

### Example 9 (Inventive)

The electrospinning is performed with the polyvinylamine solution of example 3 and the fibres are collected on a substrate until a layer is formed. The weight of the layer is determined by weighing the substrate with the layer and subtracting the weight of the substrate. Then 20 ml of the solution of 20% PAA are electrospun. During electrospinning the fibres are collected on the substrate on top of the PVAm layer until a second layer of the fibres had been formed. The substrate is weighed and the weight of the PVAm fibres determined. Then again electrospinning is performed with the polyvinylamine solution of example 3 and the fibres are collected on the substrate on top of the PAA layer until a third layer is formed. Then the weight of the third layer determined and the substrate is placed on a metal sheet and put into a 130° C. oven for 60 minutes. The ratio of the fibres in total are 50% PAA and 50% PVAm.

For each of the water-absorbent material according to examples 1 to 9 the Absorbent Capacity in saline and in water and the Desalination Index according to the above mentioned methods are determined. The results are summarized in Table 1.

TABLE 1

| | | Absorbent | Absorbent | | PAA/PVA | Fiber |
|---|---|---|---|---|---|---|
| | | Capacity (g/g) | Capacity (g/g) | Desalination | wt ratio | Diameter |
| Examples | | Deionized water | Saline (0.9%) | Index (DI) | wt % | μm |
| 9 | PVAm/PAA/PVAm 3 layers | 17.07 | 19.34 | 1.13 | ~50/50 | 0.3-3.0 |
| 8 | PAA/PVAm/PVAm 3 layers | 15.97 | 19.03 | 1.19 | ~50/50 | 0.3-3.0 |
| 7 | PAA/PVAm 2 layers* | 14.34 | 12.36 | 0.86 | ~65/35 | 0.3-3.0 |
| 6 | PAA/PVAm E-spun fiber mixing | 17.92 | 18.1 | 1.01 | ~50/50 | 0.5-3.0 |
| 5 | MDC particles | 22.21 | 17.24 | 0.78 | ~50/50 | 150-600 |
| 4 | PAA E-spun fibers | 7.6 | 5.4 | 0.71 | 100/0 | 0.3-2.0 |
| 3 | PVAm E-spun fibers | 9.4 | 6.7 | 0.71 | 0/100 | 0.3-2.0 |
| 2 | PAA E-spun fibers (DN 30) | 20.8 | 8.2 | 0.39 | 100/0 | 0.5-3.0 |
| 1 | PAA E-spun fibers (DN 50) | 28.4 | 17.3 | 0.61 | 100/0 | 0.5-3.0 |

1. Water absorbing material comprising acidic water-absorbing electrospun fibres and basic water-absorbing electrospun fibres, wherein the fibres are at least partially in contact to each other.

2. Water absorbing material according to claim 1, wherein
the acidic water-absorbing electrospun fibres comprise
at least one water-soluble acidic functional polymer and
at least one crosslinker,
and wherein
the basic water-absorbing electrospun fibres comprise
at least one water-soluble basic functional polymer and
at least one crosslinker.

3. Water absorbing material according to claim 2, wherein the water-soluble polymers are non-neutralized.

4. Water absorbing material according to claim 1, wherein the water-soluble acidic functional polymer is selected from a group consisting of polyacrylic acid, hydrolyzed starch [symbol] acrylonitrile graft copolymers, starch-acrylic acid graft copolymers, saponified vinyl acetate-acrylic esters copolymers, hydrolyzed acrylonitrile copolymers, hydro [symbol] lyzed acrylamide copolymers, ethylene-maleic anhydride copolymers, isobutylene-maleic anhydride copolymers, poly(vinylsulfonic acid), poly(vinylphosphonic acid), poly (vinylphosphoric acid), poly(vinylsulfuric acid), sulfonated polystyrene, poly(aspartic acid), poly(lactic acid), and mixtures thereof.

5. Water absorbing material according to claim 1, wherein the water-soluble basic functional polymer is selected from a group consisting of poly(vinylamine), poly(dialkylaminoalkyl(meth)acrylamide), polymer prepared from the ester analog of N(dialkylamido(meth)acrylamide), a polyethyleneiminen a poly(vinylgunidine), a poly(allylguanidine), a poly(allylamine), poly(dimethyldialkylammonium hydroxide), guanidine-modified polysterene, quternized polystyrene, quaternized poly(meth)acrylamide or ester analog thereof, poly(vinal alcohol-co-vinylamine), and mixtures thereof.

6. Water absorbing material according to according claim 1, wherein the electrospun fibres are arranged in layers.

7. Water absorbing material according to claim 6, wherein the acidic water-absorbing electro-spun fibres are arranged in at least one acidic layer and the basic water-absorbing electrospun fibres are arranged in at least one basic layer and the layers are at least partially overlapping.

8. Water absorbing material according to claim 7, wherein the basic- and acidic-layers are arranged according to a pattern.

9. Water absorbing material according to claim 8, wherein the pattern comprises basic- and acidic-layers arranged alternating.

10. Water absorbing material according to claim 1, wherein the weight ratio of acidic water-absorbing electrospun fibres to basic water-absorbing electrospun fibres is of about 95:5 to about 5:95.

11. Water absorbing material according to claim 1, wherein the thickness of each layer is not greater than 5 μm.

12. Water absorbing material according to claim 1, wherein the Desalination Index (DI) of the material is at least 0.85.

13. Water absorbing material according to claim 1 having an Absorbent Capacity in saline of at least 10 g/g.

14. Process for preparing a water absorbing material according to claim 1 comprising
preparing at least one layer of electrospun fibres by
a) electrospinning a solution of at least one acidic water-soluble polymer or of at least one basic water-soluble polymer and at least one crosslinker,
b) collecting the fibres on a substrate,
c) optionally repeating a) to b) at least once
d) crosslinking the fibres
e) optionally repeating a) to d).

15. (canceled)

16. (canceled)

17. Process according to claim 14, wherein step d) is performed by heating for a thermal sensitive crosslinker, by UV light, a pH-change, or redox.

18. A fluid absorbent core comprising water absorbing material according to claim 1.

19. A fluid absorbent article according to claim 18, comprising a core containing about 0.1% to 100% by weight of the water-absorbing material.
